(19) United States
(12) Plant Patent Application Publication
(10) Pub. No.: US 2003/0056257 P1
(43) Pub. Date: Mar. 20, 2003

(54) Clematis plant named 'Zoin'

(76) Inventor: Wim Snoeijer, Gouda (NL)

Correspondence Address: MARK P. BOURGEOIS, P.O. BOX 95, OSCEOLA, IN, 46561, US

(21) Appl. No.: 09/956,455

(22) Filed: Sep. 19, 2001

(51) Int. Cl. A01H 5/00

(52) U.S. Cl. PLT/228

(57) ABSTRACT

A new cultivar of Clematis named ‘Zoin’ that is characterized by a semi-climbing habit, low to medium height and pink flowers that are suitable for use as cut flowers. In combination these traits set ‘Zoin’ apart from all other existing varieties of Clematis known to the inventor.

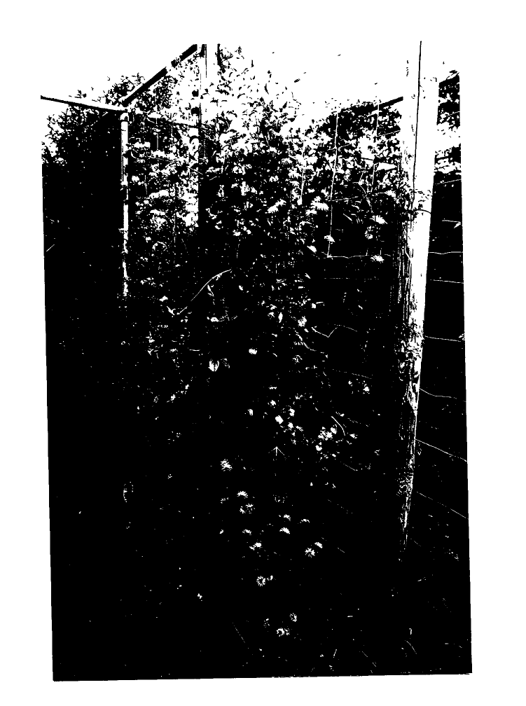

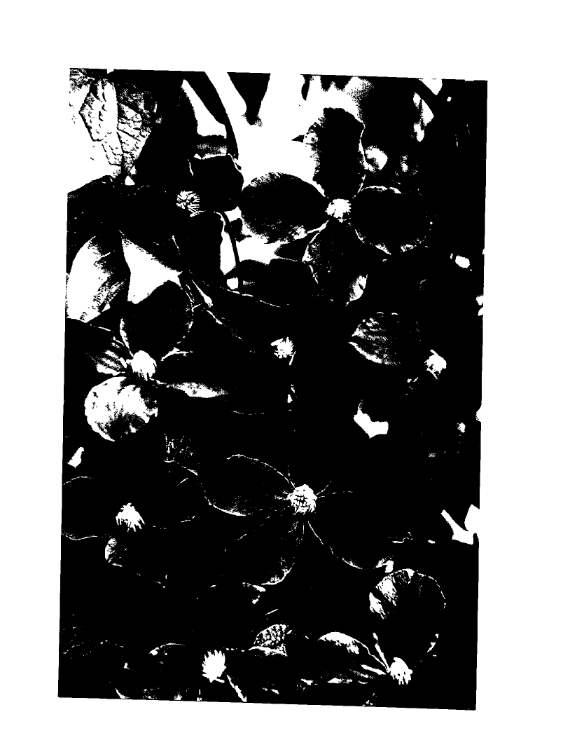

### BACKGROUND OF THE INVENTION

The present invention relates to a new and distinct cultivar of Clematis, a woody semi-climber that is grown for use in small gardens and for its cut flowers. The new cultivar is known botanically as Clematis and will be referred to hereinafter by the cultivar name ‘Zoin’.

‘Zoin’ was bred by the inventor in 1993 in a cultivated area of Boskoop, Netherlands by crossing the female parent Clematis integrifolia ‘Rosea’ with the male parent Clematis ‘Warsawska Nike’. The breeder's goal was to produce a pink form of Clematis ‘Durandii’. In addition, the breeder sought to produce a low-growing Clematis that is resistant to any disease as much as is possible and that produces long lasting cut flowers. Only one cross was made. There were no intermediate crosses. ‘Zoin’ was selected by the inventor in 1997 from the many resulting seedlings. Its pink flower with contrasting yellow stamens is a unique to the Diversifolia Group.

The distinguishing characteristics of ‘Zoin’ are its semi-upright habit, pink, open, bell shaped flowers that are more or less upright, long flowering, low to medium height and blossoms that are suitable for use as cut flowers. The closest comparison plant is Clematis ‘Durandii’ (unpatented) which exhibits a blue flower. ‘Zoin’ is distinguishable from Clematis ‘Durandii’ by its pink flowers.

‘Zoin’ was first asexually propagated in Boskoop, Netherlands in 1998 using softwood cuttings. Since that time, under careful observation, the distinguishing characteristics have been determined stable and uniform in successive generations.

### SUMMARY OF THE INVENTION

The following represent the distinguishing characteristics of the new Clematis. In combination these traits set ‘Zoin’ apart from all other existing varieties of Clematis known to the inventor. ‘Zoin’ has not been tested under all possible conditions and phenotypic differences may be observed with variations in environmental, climatic, and cultural conditions, however, without any variance in genotype.

1. The flowers of Clematis ‘Zoin’ are suitable for use as cut flowers.

2. Clematis ‘Zoin’ is low to medium in height and suitable for a small garden.

3. Clematis ‘Zoin’ exhibits open, bell shaped, pink and more or less upright flowers..

4. Clematis ‘Zoin’ is a woody semi-climber that is hardy to USDA Zone 5.

5. Clematis ‘Zoin’ exhibits dark green foliage.

6. Clematis ‘Zoin’ exhibits a semi-upright habit.

7. Clematis ‘Zoin’ exhibits a particular long flowering period and flowers in late spring and summer.

### BRIEF DESCRIPTION OF THE DRAWINGS

The accompanying color photographs illustrate the distinguishing traits of ‘Zoin’. The plants in the photographs were grown out of doors in Boskoop, Netherlands in one-gallon containers before planting in the ground. The photograph on sheet 1 illustrates entire plants in bloom from a side perspective. The photograph on sheet 2 is a close-up view of the leaves and flowers. All photographs are taken using conventional techniques and although foliage colors may appear different from actual colors due to light reflectance, they are as accurate as possible by conventional photography.

### BOTANICAL DESCRIPTION OF THE PLANT

The following is a detailed description of the Clematis cultivar named ‘Zoin’. Data was collected in Arroyo Grande, Calif. from plants grown in onegallon containers out of doors. Color determinations are in accordance with The Royal Horticultural Society Colour Chart except where general color terms of ordinary dictionary significance are used. The growing requirements are similar to the species. Under normal growing conditions there are no known disease problems known to the inventor.

Botanical classification: Clematis ‘Zoin’ (Diversifolia Group).

Uses: Cut flowers and semi-climber for a small garden.

Parent: ‘Zoin’ is a hybrid resulting from the induced hybridization of the following plants:

Female parent.—Clematis integrifolia ‘Rosea’.

Male parent.—Clematis ‘Warsawska Nike’.

Type: Perennial.

Vigour: Vigorous. Generally grows 1.70 meters in length during the first year.

Habit: Semi-climber.

Height (one-gallon): 42 cm. in height (at maturity ‘Zoin’ is 150-200 cm. in height).

Width (one-gallon): 60 cm. in width.

Hardiness: USDA Zone 5.

Propagation: Softwood cuttings.

Root system: Fibrous.

Soil: Plant in well-drained soils.

Sunlight: Plant in full sunlight.

Plant sexuality: Bisexual.

Time to initiate roots: 2 weeks are required for an initial cutting to produce roots.

Crop time: 8-10 months are required to produce a finished one-gallon from a rooted cutting.

Seasonal interest: Flowers in late spring and summer.

Stem:

Branching habit.—Semi-upright.

Internode length.—8-10 cm. between nodes.

Stem diameter.—3 mm. in diameter.

Stem length.—40 cm. in length.

Shape.—Cylindrical and articulate.

Surface.—Fluted with pubescent nodes.

Texture.—Suffruticose with pithy center.

Stem color.—171 B.

Foliage:

Type.—Deciduous.

Leaf arrangement.—Opposite.

Leaf division.—Compound (singular and ternary).

Leaflet division.—Simple.

Leaflet shape.—Oblong to lanceolate.

Leaflet base.—Base of terminal leaflet obtuse-subcordate. Base of side leaves oblique.

Leaflet apex.—Attenuate.

Leaflet venation.—Reticulate.

Vein color (adaxial surface).—147 D.

Vein color (abaxial surface).—147 D.

Leaf surface (adaxial).—Matte and puberulent.

Leaf surface (abaxial).—Shiny and puberulent.

Leaf attachment.—Petiolate and articulate. Side leaflets are sub-sessile to short articulate.

Petiole dimensions.—2.5-6 cm. in length and 2 mm. in diameter.

Petiole color.—138 C.

Petiole shape.—Cylindrical and twisted.

Leaf margin.—Entire and revolute.

Leaf texture.—Soft and flexible.

Leaf length.—7-9 cm. in length.

Leaf width.—5-7 cm. in width.

Leaflet length.—2-4 cm. in length.

Leaflet width.—1-2 cm. in width.

Leaf color (adaxial surface).—147 A.

Leaf color (abaxial surface).—147 C.

Foliar fragrance.—Absent.

Flower:

Form.—Axillary and terminal inflorescence.

Inflorescence.—Dichasial cyme.

Persistent or self-cleaning.—Self-cleaning.

Color of peduncle.—148 D.

Peduncle length.—5.1-10 cm. in length.

Peduncle diameter.—1.1-3 mm. in diameter.

Surface of peduncle.—Pubescent.

Diameter of flower.—6-10 cm. in diameter.

Shape.—Open campanulate.

Sexuality.—Bisexual.

Aspect.—Facing upwards.

Bud.—Upright and lanceolate.

Bud color.—61 A.

Bud surface.—Glossy.

Petals.—Apetalous.

Tepals.—Four in number.

Color of tepals (inside surface).—A combination of 72 C suffused with 67 A.

Color of tepals (outside surface).—72 C.

Tepal shape.—Oblanceolate-obovate.

Tepal apex.—Revolute and mucronate.

Tepal surface.—Furrowed.

Tepal margin.—Sinuous and pubescent.

Tepal length.—5 cm. in length.

Tepal width.—3 cm. in width.

Unfused or fused.—Tepals are unfused and do not overlap.

Blooming period.—End of June, July, August and the beginning of September.

Fragrance.—Slightly fragrant.

Reproduction organs:

Stamens.—Polyandrous.

Pubescence.—Present.

Arrangement of stamens.—Spirally arranged.

Color of stamen filament.—12 B.

Dimensions of stamen.—0.5-0.8 cm. in length and 1 mm in diameter.

Dimensions of anther.—1 mm. in width and 0.4 cm. in length.

Pollen color.—4 D.

Anther color.—4 D to 151 A and becoming black 202 A.

Pistil.—One.

Color of pistil.—155 C.

Dimensions of pistil.—0.9-1 cm.

Pubescence.—Present.

Stigma color.—155 C.

Style color.—155 C.

Style form.—Plumose.

Ovary position.—Superior.

Ovary color.—Pale brown.

Ovary shape.—Oval.

Seed:

Dimensions of fruit.—4 cm. in diameter and 2 cm. in height.

Seed surface.—Villous.

Villi color.—156 C.

Seed shape.—Oval.

Seed color.—136 A.

Seed dimensions.—1.50 mm. in diameter and 3 mm. in length.

Style dimensions.—2 cm. in length and 3-4 mm. in width.

Style shape.—Seculate.

Style form.—Plumose.

Style color.—136 A.

1. A new and distinct variety of clematis plant named ‘Zoin’ as described and illustrated:
